(19) United States
(12) Patent Application Publication
(10) Pub. No.: US 2016/0317091 A1
(43) Pub. Date: Nov. 3, 2016

(54) Prenatal Monitor Assembly

(71) Applicants: Olasinbo Olukoya, Hillsborough, NJ (US); Seyi Okuwa, Richmond, TX (US)

(72) Inventors: Olasinbo Olukoya, Hillsborough, NJ (US); Seyi Okuwa, Richmond, TX (US)

(21) Appl. No.: 14/699,274

(22) Filed: Apr. 29, 2015

(51) Int. Cl. A61B 5/00; A61B 5/0205

(52) U.S. Cl. CPC A61B 5/6831; A61B 5/746; A61B 5/4356; A61B 5/0205; A61B 5/0002; A61B 5/02411

(57) ABSTRACT

A prenatal monitor assembly for monitoring an in utero babys' heart rate and uterine contractions. The assembly includes a belt that may be worn around a pregnant woman's abdomen. A first coupler is attached to the belt and a second coupler is attached to the belt. Each of the first coupler and the second coupler are complementary wherein the first coupler and the second coupler may retain the belt around the pregnant woman's abdomen. A monitor is attached to the belt to monitor a heart beat of the in utero baby and uterine contractions. A panel is provided. A primary coupler is coupled to the panel and a secondary coupler is coupled to the panel. The primary coupler is complementary with respect to the first coupler and the secondary coupler is complementary with respect to the second coupler wherein the panel increases a diameter of the belt.

### BACKGROUND OF THE DISCLOSURE

### Field of the Disclosure

The disclosure relates to monitor devices and more particularly pertains to a new monitor device for monitoring an in utero baby's heart rate and uterine contractions.

### SUMMARY OF THE DISCLOSURE

An embodiment of the disclosure meets the needs presented above by generally comprising a belt that may be worn around a pregnant woman's abdomen. A first coupler is attached to the belt and a second coupler is attached to the belt. Each of the first coupler and the second coupler are complementary wherein the first coupler and the second coupler may retain the belt around the pregnant woman's abdomen. A monitor is attached to the belt to monitor a heart beat of the in utero baby and uterine contractions. a panel is provided. A primary coupler is coupled to the panel and a secondary coupler is coupled to the panel. The primary coupler is complementary with respect to the first coupler and the secondary coupler is complementary with respect to the second coupler wherein the panel increases a diameter of the belt.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a prenatal monitor assembly according to an embodiment of the disclosure.

FIG. 2 is a back view of an embodiment of the disclosure.

FIG. 3 is a top view of an embodiment of the disclosure.

FIG. 4 is a cross sectional view taken along line 4-4 of FIG. 3 of an embodiment of the disclosure.

FIG. 5 is a perspective in-use view of an embodiment of the disclosure.

FIG. 6 is a schematic view of an embodiment of the disclosure.

FIG. 7 is a schematic view of an alternative embodiment of the disclosure.

### DESCRIPTION OF THE PREFERRED EMBODIMENT

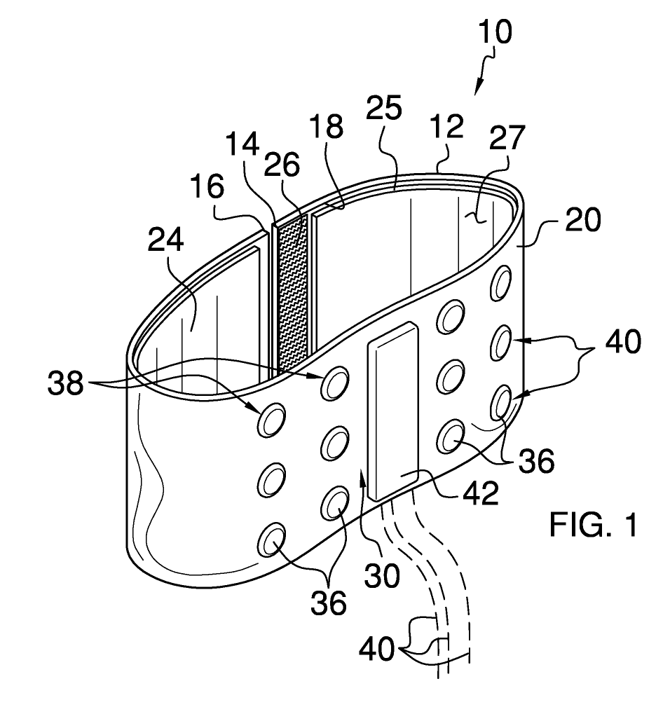

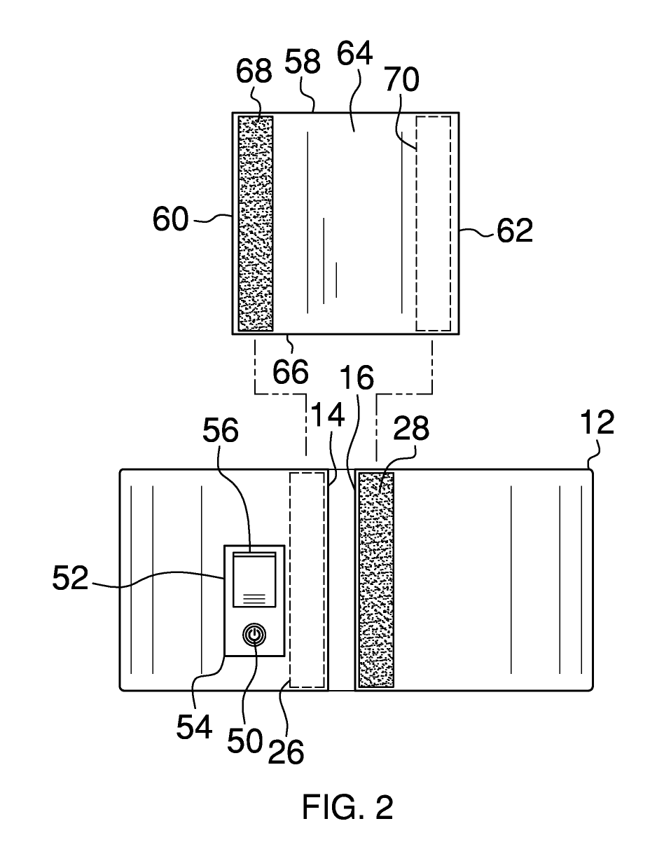

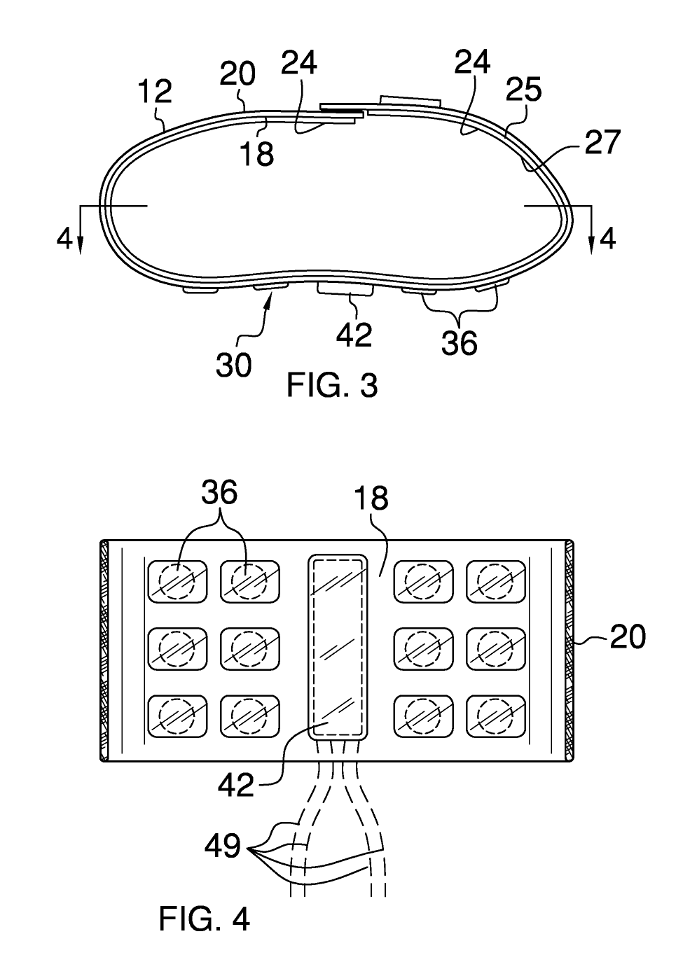

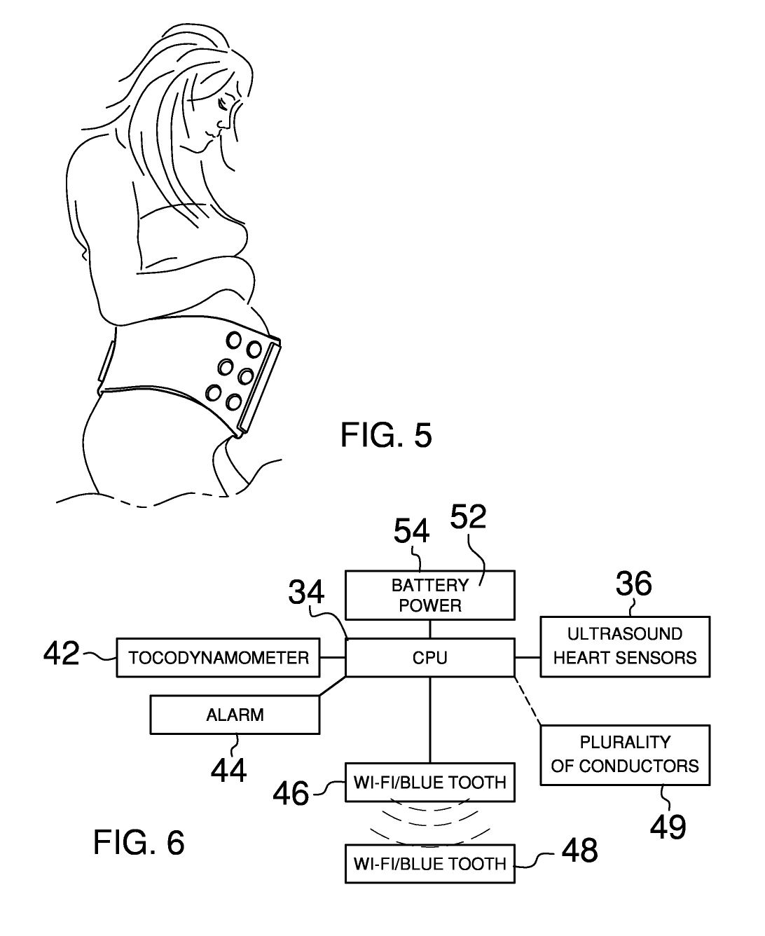

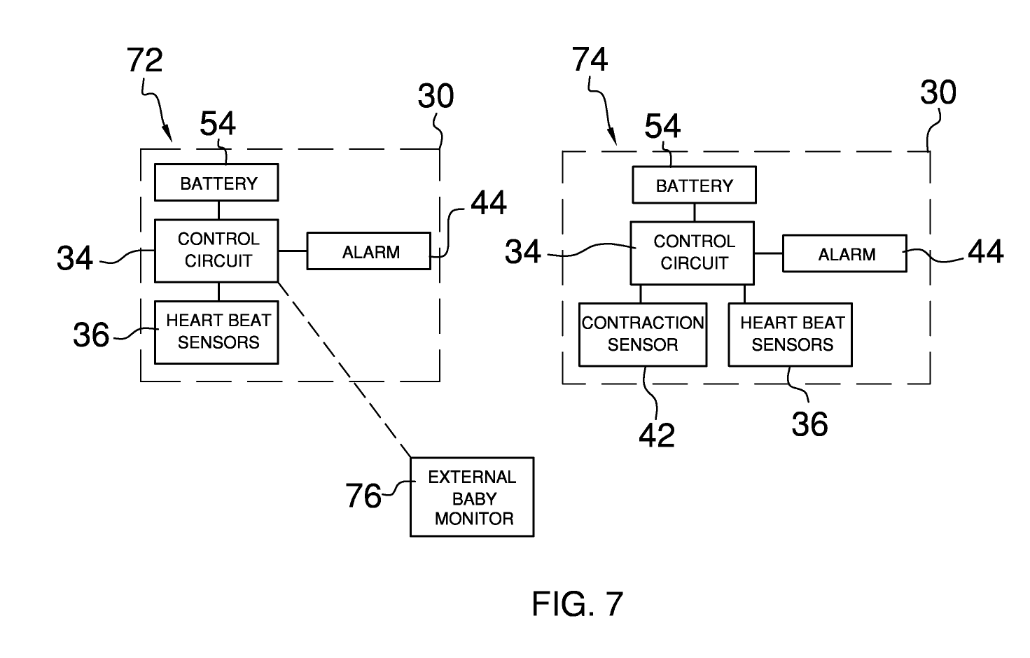

With reference now to the drawings, and in particular to FIGS. 1 through 7 thereof, a new monitor device embodying the principles and concepts of an embodiment of the disclosure and generally designated by the reference numeral 10 will be described.

As best illustrated in FIGS. 1 through 7, the prenatal monitor assembly 10 generally comprises a belt 12 that has a first end 14, a second end 16, a back side 18 and a front side 20. The belt 12 is elongated between the first end 14 and the second end 16 wherein the belt 12 may be worn around an abdomen 22 of a pregnant woman 24. The belt 12 may be comprised of a resiliently stretchable material. A lining 24 is provided that has a front surface 25 and a back surface 27 and the front surface 25 is removably attached to the back side 18 of the belt 12. The lining 24 is substantially coextensive with the belt 12 and the back surface 27 may abut the pregnant woman's abdomen 22. The lining 24 may be comprised of a resiliently stretchable material.

A first coupler 26 is attached to the belt 12. The first coupler 26 is positioned on the back side 18 and the first coupler 26 is positioned adjacent to the first end 14. A second coupler 28 is attached to the belt 12. The second coupler 28 is positioned on the front side 20 and the second coupler 28 is positioned adjacent to the second end 16. Each of the first coupler 26 and the second coupler 28 is complementary wherein the first coupler 26 and the second coupler 28 retain the belt 12 around the pregnant woman's abdomen 22. Each of the first coupler 26 and the second coupler 28 may comprise a hook and loop fastener.

A monitor 30 is attached to the belt 12 wherein the monitor 30 may monitor a heart beat of an in utero baby 32 and uterine contractions. The monitor 30 comprises a control circuit 34 that is attached to the belt 12. The control circuit 34 may comprise an electronic processor or the like. A plurality of heart beat sensors 36 is attached to the belt 12 wherein each of the heart beat sensors 36 may detect the heart beat of the in utero baby 32. Each of the heart beat sensors 36 is positioned on the front side 20 and the heart beat sensors 36 are arranged in a plurality of columns 38 and rows 40 on the front side 20. Each of the heart beat sensors 36 is electrically coupled to the control circuit 34 and each of the heart beat sensors 36 may comprise a Doppler heart beat monitor of any conventional design. The plurality of heat beat sensors 36 allow the in utero baby's 32 heart beat to be monitored regardless of where the in utero baby 32 is positioned within the pregnant woman's abdomen 22.

A contraction sensor 42 is attached to the belt 12 to detect uterine contractions. The contraction sensor 42 is positioned on the front side 20 and the contraction sensor 42 is electrically coupled to the control circuit 34. The contraction sensor 42 may comprise a tocodynamometer of any conventional design. An alarm 44 is attached to the belt 12 and the alarm 44 is electrically coupled to the control circuit 34. The alarm 44 generates an alarm sequence when the heart beat sensors 36 detects a heart beat that is below a trigger heart rate and when the contraction sensor 42 detects a uterine contraction that is above a trigger contraction rate. The trigger heart rate may be approximately 120 beats per minute and the trigger contraction rate may be uterine contractions that have a duration that is greater than approximately one minute and occur less than approximately every five minutes.

The alarm 44 includes a transmitter 46 that is attached to the belt and the transmitter 46 is electrically coupled to the control circuit 34. The transmitter 46 is in communication with an external electronic device 48 such that the in utero baby's 32 heart beat and the uterine contractions may be observed on the external electronic device 48. The transmitter 46 transmits the alarm sequence to the external electronic device 48. The transmitter 46 may be a radio frequency transmitter and the external electronic device 48 may be a personal computer or the like. Alternatively, a plurality of conductors 49 may be provided and each of the conductors 49 may be electrically coupled to the control circuit 34. Each of the conductors 49 may be electrically coupled to the external electronic device 48 thereby facilitating the in utero baby's 32 heart beat and the uterine contractions to be observed on the external electronic device 48.

A switch 50 is attached to the belt 12 and the switch 50 is positioned on the front side 20. The switch 50 is positioned adjacent to the first end 14. The switch 50 is electrically coupled to the control circuit 34 and the switch 50 selectively actuates and de-actuates the control circuit 34. A power supply 52 is attached to the belt 12. The power supply 52 is positioned on the front side 20 and the power supply 52 is positioned adjacent to the first end 14. The power supply 52 is electrically coupled to the switch 50 and the power supply 52 comprises at least one battery 54. The switch 50 may be positioned on the power supply 52. A battery cover 56 is removably coupled to the power supply 52 to access an interior of the power supply 52.

A panel 58 is provided that has a first end 60, a second end 62, a front side 64 and a back side 66. The panel 58 may be comprised of a resiliently stretchable material. A primary coupler 68 is coupled to the front side 64 of the panel 58 and the primary coupler 68 is positioned adjacent to the first end 60 of the panel 58. A secondary coupler 70 is coupled to the back side 66 of the panel 58 and the secondary coupler 70 is positioned adjacent to the second end 62 of the panel 58. The primary coupler 68 is complementary with respect to the first coupler 26 and the secondary coupler 70 is complementary with respect to the second coupler 28 wherein the panel 58 increases a diameter of the belt 12 thereby facilitating the belt 12 to be worn by pregnant women 24 of various sizes. Each of the primary 68 and secondary 70 couplers may comprise hook and loop fasteners.

Alternatively, the monitor 30 may be offered in a home version 72 that utilizes only the heart beat sensors 36 and does not utilize the contraction sensor 42. The monitor may be offered in a hospital version 74 that utilizes the heart beat sensors 36 and also utilizes the contraction sensor 42. The home version 72 of the monitor 30 may be electrically coupled to an external baby monitor 76 or the like thereby allowing the in utero baby's 32 heart beat to be heard by an observer.

In use, the belt 12 is worn around the pregnant woman's abdomen 22 in order to detect the in utero baby's 32 heart rate and the uterine contractions. The external electronic device 48 is observed to monitor the in utero baby's 32 heart rate and the uterine contractions. The external electronic device 48 issues an audible warning when the alarm 44 generates the alarm sequence such that the pregnant woman 24 is advised to seek medical attention.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A prenatal monitor assembly configured to monitor a baby's heartbeat while the baby is in utero, said assembly comprising:
a belt being configured to be worn around a pregnant woman's abdomen;
a first coupler being attached to said belt;
a second coupler being attached to said belt, each of said first coupler and said second coupler being complementary wherein said first coupler and said second coupler are configured to retain said belt around the pregnant woman's abdomen;
a monitor being attached to said belt wherein said monitor is configured to monitor a heart beat of the in utero baby and uterine contractions.
a panel;
a primary coupler being coupled to said panel; and
a secondary coupler being coupled to said panel, said primary coupler being complementary with respect to said first coupler and said secondary coupler being complementary with respect to said second coupler wherein said panel increases a diameter of said belt.

2. The assembly according to claim 1, wherein said belt has a first end, a second end, a back side and a front side, said belt being elongated between said first end and said second end.

3. The assembly according to claim 2, further comprising a lining having a front surface and a back surface, said front surface being removably attached to said back side of said belt, said lining being substantially coextensive with said belt, said back surface being configured to abut the pregnant woman's abdomen.

4. The assembly according to claim 2, wherein said first coupler is positioned on said back side, said first coupler being positioned adjacent to said first end.

5. The assembly according to claim 2, wherein said second coupler is positioned on said front side, said second coupler being positioned adjacent to said second end.

6. The assembly according to claim 1, wherein said monitor comprises a control circuit being attached to said belt.

7. The assembly according to claim 6, further comprising:
said belt having a front side; and
a plurality of heart beat sensors being attached to said belt wherein each of said heart beat sensors is configured to detect the heart beat of the in utero baby, each of said heart beat sensors being positioned on said front side, said heart beat sensors being arranged in a plurality of columns and rows on said front side, each of said heart beat sensors being electrically coupled to said control circuit.

8. The assembly according to claim 7, further comprising a contraction sensor being attached to said belt wherein said contraction sensor is configured to detect uterine contractions, said contraction sensor being positioned on said front side, said contraction sensor being electrically coupled to said control circuit.

9. The assembly according to claim 6, further comprising an alarm being attached to said belt, said alarm being electrically coupled to said control circuit, said alarm generating an alarm sequence when said heart beat sensors detects a heart beat being below a trigger heart rate and said contraction sensor detects a uterine contraction being above a trigger contraction rate.

10. The assembly according to claim 9, further comprising a transmitter being attached to said belt, said transmitter being electrically coupled to said control circuit, said transmitter being configured to be in communication with an external electronic device such that the in utero baby's heart beat and the uterine contractions may be observed on the external electronic device, said transmitter transmitting said alarm sequence to the external electronic device.

11. The assembly according to claim 6, further comprising:
said belt having a front side and a first end; and
a switch being attached to said belt, said switch being positioned on said front side, said switch being positioned adjacent to said first end, said switch being electrically coupled to said control circuit, said switch selectively actuating and de-actuating said control circuit.

12. The assembly according to claim 11, wherein a power supply being attached to said belt, said power supply being positioned on said front side, said power supply being positioned adjacent to said first end, said power supply being electrically coupled to said switch, said power supply comprising at least one battery.

13. The assembly according to claim 1, wherein said panel has a first end, a second end, a front side and a back side, said primary coupler being positioned adjacent to said first end of said panel, said secondary coupler being positioned adjacent to said second end of said panel.

14. A prenatal monitor assembly configured to monitor a baby's heartbeat while the baby is in utero, said assembly comprising:
a belt having a first end, a second end, a back side and a front side, said belt being elongated between said first end and said second end wherein said belt is configured to be worn around a pregnant woman's abdomen;
a lining having a front surface and a back surface, said front surface being removably attached to said back side of said belt, said lining being substantially coextensive with said belt, said back surface being configured to abut the pregnant woman's abdomen;
a first coupler being attached to said belt, said first coupler being positioned on said back side, said first coupler being positioned adjacent to said first end;
a second coupler being attached to said belt, said second coupler being positioned on said front side, said second coupler being positioned adjacent to said second end, each of said first coupler and said second coupler being complementary wherein said first coupler and said second coupler are configured to retain said belt around the pregnant woman's abdomen;
a monitor being attached to said belt wherein said monitor is configured to monitor a heart beat of the in utero baby and uterine contractions, said monitor comprising
a control circuit being attached to said belt,
a plurality of heart beat sensors being attached to said belt wherein each of said heart beat sensors is configured to detect the heart beat of the in utero baby, each of said heart beat sensors being positioned on said front side, said heart beat sensors being arranged in a plurality of columns and rows on said front side, each of said heart beat sensors being electrically coupled to said control circuit,
a contraction sensor being attached to said belt wherein said contraction sensor is configured to detect uterine contractions, said contraction sensor being positioned on said front side, said contraction sensor being electrically coupled to said control circuit,
an alarm being attached to said belt, said alarm being electrically coupled to said control circuit, said alarm generating an alarm sequence when said heart beat sensors detects a heart beat being below a trigger heart rate and said contraction sensor detects a uterine contraction being above a trigger contraction rate,
a transmitter being attached to said belt, said transmitter being electrically coupled to said control circuit, said transmitter being configured to be in communication with an external electronic device such that the in utero baby's heart beat and the uterine contractions may be observed on the external electronic device, said transmitter transmitting said alarm sequence to the external electronic device,
a switch being attached to said belt, said switch being positioned on said front side, said switch being positioned adjacent to said first end, said switch being electrically coupled to said control circuit, said switch selectively actuating and de-actuating said control circuit, and
a power supply being attached to said belt, said power supply being positioned on said front side, said power supply being positioned adjacent to said first end, said power supply being electrically coupled to said switch, said power supply comprising at least one battery;
a panel having a first end, a second end, a front side and a back side;
a primary coupler being coupled to said front side of said panel, said primary coupler being positioned adjacent to said first end of said panel; and
a secondary coupler being coupled to said back side of said panel, said secondary coupler being positioned adjacent to said second end of said panel, said primary coupler being complementary with respect to said first coupler and said secondary coupler being complementary with respect to said second coupler wherein said panel increases a diameter of said belt.
